(19) United States
(12) Patent Application Publication
(10) Pub. No.: US 2020/0392566 A1
(43) Pub. Date: Dec. 17, 2020

(54) Primer, And Kit For Rapid Detection Of Bacteria And Application Thereof

(71) Applicant: Shanghai East Hospital (East Hospital Affiliated to Tongji University), Shanghai (CN)

(72) Inventors: Zhongmin LIU, Shanghai (CN); Wenwen JIA, Shanghai (CN); Zhihui BAI, Shanghai (CN); Hongming TANG, Shanghai (CN)

(73) Assignee: Shanghai East Hospital (East Hospital Affiliated to Tongji University), Shanghai (CN)

(21) Appl. No.: 16/673,021

(22) Filed: Nov. 4, 2019

(30) Foreign Application Priority Data
Jun. 13, 2019 (CN) 201910510071.9

(51) Int. Cl. C12Q 1/689

(52) U.S. Cl. CPC C12Q 1/689

(57) ABSTRACT

The present disclosure relates to a primer and a kit for rapid detection of bacteria and an application thereof, belonging to the technical field of bacterial detection. The present disclosure provides a primer for rapid detection of bacteria, a nucleotide sequence of a forward primer for the detection is shown in SEQ ID NO.1, and a nucleotide sequence of a reverse primer is shown in SEQ ID NO.2; a degenerate base W=A/T in the forward primer, and a degenerate base R=A/G in the reverse primer. The primer of the present disclosure may achieve rapid, wide, high-sensitivity and quantitative detection of bacteria, can be applied in the field of bacterial detection, and is more suitable for the clinical research and application of cells and stem cell-based products.

### CROSS-REFERENCE TO RELATED APPLICATIONS

This application claims priority to Chinese application number 201910510071.9, filed Jun. 13, 2019 with a title of Primer, And Kit For Rapid Detection Of Bacteria And Application Thereof. The above-mentioned patent application is incorporated herein by reference in its entirety.

### FIELD

The present disclosure relates to the technical field of bacterial detection, and in particular to a primer, and a kit for rapid detection of bacteria and application thereof.

### BACKGROUND

Bacteria have a wide distribution in nature, numerous varieties, rapid reproduction and small volume, and cause cell contamination in clinical research and application easily, bringing severe damage. Antibiotics may prevent contamination and infection, but may not inhibit the growth of bacteria completely; therefore the real-time detection of bacteria is particularly important in clinical research.

Culture method is a main sterile detection way in Chinese Pharmacopoeia, and as a standard detection method of sterile detection, the culture method has high accuracy, but it usually takes 14 d to get the result of the detection report, and takes longer to the slowly-growing bacteria. There is a rather high demand for the survival rate and growth activity of the cells applied clinically, and the clinical efficacy of cells will be affected after waiting for the result of detection for a long time, especially for the clinical application of stem cells. Guidelines for Quality Control and Preclinical Study of Stem Cell-based Preparations has stipulated ten major detection items of stem cells, including sterility test, and indicated that the releasing detection items should reflect the quality and safety information of cell preparations within shorter period of time. Therefore, to explore a method for detecting bacteria rapidly is a problem to be solved urgently in clinical research. At present, a full-automatic microorganism biochemical identification system, e.g., BioMerieux, etc. has been used in the detection of bacteria to obtain a result according to the growth changes and chromogenic reaction in microbial pores, the method is easy to operate, but requires high cost of instrument, moreover, it takes 5 d to obtain the result of the report usually. Currently, there is a commercial strip for rapid test as well, the method only needs to culture cells for 24 h, but it is easy to cause false negative results. As for the use of specific enzymes, mass-spectrometric technique, immunosensor and immune-magnetic separation technology, it has high sensitivity to specific bacteria but poor universality, not applicable to the detection of unknown bacteria.

### SUMMARY

An objective of the present disclosure is to provide a primer and a kit for rapid bacterial detection. The primer of the present disclosure may achieve rapid, wide, high-sensitivity and quantitative detection of bacteria, and may be applied in cell products, especially in the clinical detection and research of stem cell-based products.

The present disclosure provides a primer for rapid detection of bacteria, a nucleotide sequence of a forward primer for the detection is shown in SEQ ID NO.1, and a nucleotide sequence of a reverse primer is shown in SEQ ID NO.2;

a degenerate base W=A/T in the forward primer, and a degenerate base R=A/G in the reverse primer.

The present disclosure further provides a kit for rapid detection of bacteria, and the kit includes the primer in the above technical solution and a standard.

Preferably, the standard includes a plasmid pUC57 containing a sequence of a 16S rDNA conserved region, and a nucleotide sequence of the sequence of the 16S rDNA conserved region is shown in SEQ ID NO.3.

Preferably, the service concentration of the primer is 100-400 nM.

Preferably, Q-PCR reaction conditions of the kit are: 95° C., 10 min; 95° C., 20 s, 58° C., 30 s, 72° C., 30 s, 40 cycles; 95° C., 15 s, 60° C., 1 min, 95° C., 15 s, 60° C., 15 s.

Preferably, conventional PCR reaction conditions of the kit are: 95° C., 10 min; 95° C., 20 s, 58° C., 30 s, 72° C., 30 s, 35 cycles; 72° C., 5 min.

The present disclosure further provides an application of the kit of the above technical solution in the detection of bacteria.

The present disclosure further provides an application of the kit of the above technical solution in the detection of bacteria of cells.

The present disclosure further provides an application of the kit of the above technical solution in the detection of bacteria of stem cells.

The present disclosure provides a primer for rapid detection of bacteria. The primer provided by the present disclosure may achieve the detection of bacteria by means of nucleic acid amplification. Compared with the traditional bacterial detection (in need of 14 d at least), the primer of the present disclosure may achieve the detection of bacteria within 2-3 h. The primer of the present disclosure may detect all strains containing upstream and downstream primers, including Escherichia coli, Staphylococcus aureus, Bacillus subtilis, Pseudomonas aeruginosa, Salmonella, streptococcus, degenerating Bacilus, Klebsiella pneumoniae, etc. The primer of the present disclosure may achieve rapid, wide, high-sensitivity and quantitative detection of bacteria, and may be applied in cell products, especially in the clinical detection and research of stem cell-based products.

### BRIEF DESCRIPTION OF THE DRAWINGS

FIG. 1 is a standard curve provided by the present disclosure;

FIG. 2 is a diagram showing detection results of strains by conventional PCR provided by the present disclosure;

FIG. 3 is a diagram showing detection results of human and murine cells by conventional PCR provided by the present disclosure.

### DETAILED DESCRIPTION

The The present disclosure provides a primer for rapid detection of bacteria, a nucleotide sequence of a forward primer for the detection is shown in SEQ ID NO.1, and a nucleotide sequence of a reverse primer is shown in SEQ ID NO.2;

a degenerate base W=A/T in the forward primer, and a degenerate base R=A/G in the reverse primer.

There is no specific limitation to the source of the primer of the present disclosure as long as a conventional artificial synthesis method well known to those skilled in the art is taken available, e.g., it is synthesized by Shanghai Generay Biotech Co., Ltd. The specific nucleotide sequence of the forward primer of the present disclosure is 5′-CGAWGCAACGCGAAGAACC-3′; the reverse primer: 5′-CACCTTCCTCCRGTTTRT-3′. By BLAST and BioEdit software, the present disclosure makes contrastive analysis on the sequence of the bacterial 16S rDNA conserved region, and primers are designed according to the sequence. The primer of the present disclosure may detect all strains containing upstream and downstream primers, including various common bacteria, e.g., Escherichia coli, Staphylococcus aureus, Bacillus subtilis, Pseudomonas aeruginosa, Salmonella, streptococcus, degenerating Bacilus, Klebsiella pneumoniae, etc.; moreover, the difference of TM from the standard is not more than 2° C., and the difference of TM among bacteria is not more than 2° C. The primer of the present disclosure has good specificity, and negative detection results to the gene sequence of non-bacterial species, e.g., human immune cells, rat cells. The primer of the present disclosure may detect the bacterial contamination condition of multiple strains mixture according to the actual situation.

The present disclosure further provides a kit for rapid detection of bacteria, and the kit includes the primer in the above technical solution and standard. In the present disclosure, the standard includes a plasmid pUC57 containing a sequence of a 16S rDNA conserved region, and a nucleotide sequence of the sequence of the 16S rDNA conserved region is shown in SEQ ID NO.3:

ATTCGATGCAACGCGAAGAACCTTACCTGGTCTTGACATCCACAGAAC
TTTCCAGAGATGGATTGGTGCCTTCGGGAACTGTGAGACAGGTGCTGC
ATGGCTGTCGTCAGCTCGTGTTGTGAAATGTTGGGTTAAGTCCCGCAA
CGAGCGCAACCCTTATCCTTTGTTGCCAGCGGTCCGGCCGGGAACTCA
AAGGAGACTGCCAGTGATAAACTGGAGGAAGGTGGGG.

In the present disclosure, the nucleotide sequence of the sequence of the 16S rDNA conserved region is synthesized by Shanghai Generay Biotech Co., Ltd. In the present disclosure, the standard is preferably diluted by 1×TE buffer. There is no special limitation to other components of the kit in the present disclosure as long as conventional Q-PCR and other common PCR components are taken available, it is purchased from Promega company preferably when Q-PCR detection is conducted, A6002 product is matched with the primer and standard of the present disclosure; it is purchased from TIANGEN company preferably when conventional PCR detection is conducted, KT205 product is matched with the primer and standard of the present disclosure. The standard is preferably used to build a standard curve in the present disclosure, and the scope of DNA copy number capable of being detected by the present disclosure is within 1.2×10²-3×10⁷ copies. In the present disclosure, the service concentration of the primer is 100-400 nM. The higher the primer concentration is, the better the amplification efficiency is, while the specificity is slightly poor, and the optimal concentration needs to be adjusted according to different instrument and reagents. In the present disclosure, Q-PCR reaction conditions of the kit are: 95° C., 10 min; 95° C., 20 s, 58° C., 30 s, 72° C., 30 s, 40 cycles; 95° C., 15 s, 60° C., 1 min, 95° C., 15 s, 60° C., 15 s. The amplification efficiency of the Q-PCR reaction in the present disclosure is 86%, R2 is 0.9971. In the present disclosure, conventional PCR reaction conditions of the kit are: 95° C., 10 min; 95° C., 20 s, 58° C., 30 s, 72° C., 30 s, 35 cycles; 72° C., 5 min. In the present disclosure, the conventional PCR reaction system is preferably 20 μl, where the forward primer is 100-400 nM and the reverse primer is 100-400 nM. In the present disclosure, as for the conventional PCR, 2% agarose gel electrophoresis is preferably used for detection (120 V, 20 min), and DL2000 is Marker. In the present disclosure, the sample to be detected is preferably pretreated before detection, and the pretreatment method is to directly process the sample for 10 min at 100° C. preferably without RNA extraction and inverse transcription. In the present disclosure, the sample to be detected is preferably supernatant with small dosage, and only 1 μl sample may be used for detection.

When Q-PCR is taken for detection, if CT value of the QPCR sample is >35 or the CT value is <35, but the difference of TM value from the positive standard is >2° C., it is negative; in conventional PCR, if no strip is found at 229 bp, it is negative.

The present disclosure further provides an application of the kit of the above technical solution in the detection of bacteria.

The present disclosure further provides an application of the kit of the above technical solution in sterile detection of bacteria of cells.

The present disclosure further provides an application of the kit of the above technical solution in sterile detection of bacteria of stem cells.

The primer and kit for rapid detection of bacteria and application thereof in the present disclosure will be further described in detail with reference to specific examples, and the technical solution of the present disclosure includes but not limited to the following examples.

### Example 1

I. Preparation of the Primer and Standard

1. A forward primer of the present disclosure: 5′-CGAWGCAACGCGAAGAACC-3′; a reverse primer: 5′-CACCTTCCTCCRGTTTRT-3′. The primer were synthesized by Shanghai Generay Biotech Co., Ltd., 12000 g samples were centrifuged for 1 min, forward and reverse primers were dissolved to concentration of 100 μM by adding water, 2 μl were taken and added to 18 μl water to be diluted to 10 μM, the rest primer was put into a −20° C. refrigerator for preservation.

2. The standard of the present disclosure is a plasmid pUC57 containing the sequence of the 16S rDNA conserved region, and synthesized by Shanghai Generay Biotech Co., Ltd. PUC57 base number was 2710 bp. The sequence of the 16S rDNA conserved region:

ATTCGATGCAACGCGAAGAACCTTACCTGGTCTTGACATCCACAGAAC
TTTCCAGAGATGGATTGGTGCCTTCGGGAACTGTGAGACAGGTGCTGC
ATGGCTGTCGTCAGCTCGTGTTGTGAAATGTTGGGTTAAGTCCCGCAA
CGAGCGCAACCCTTATCCTTTGTTGCCAGCGGTCCGGCCGGGAACTCA
AAGGAGACTGCCAGTGATAAACTGGAGGAAGGTGGGG,

and the base number was 229 bp. The standard was dissolved by 1×TE buffer to make concentration of 216.7 ng/μl, and the copy number was counted as 6.6×10¹⁰ copies/μl.

II. Preparation of a Standard Curve and Sensitivity Detection

1. Dilution of standard: a 200 μl PCR tube was taken and added 1×TE buffer-diluted standard. The standard was diluted in gradient by 10 times from stock solution to obtain 12 concentrations.

2. Preparation of QPCR system: total volume of the reaction system was 20 μl, the forward primer was 300 nM, reverse primer was 300 nM and the template was 1 μl. The reaction conditions were divided into three stages, the first one was a predegeneration stage for 10 min at 95° C., the second one was a PCR reaction stage with 40 cycles for 20 s, 30 s and 30 s at 95° C., 58° C. and 72° C. respectively; the third one was a dissolving stage of amplified products for 15 s, 1 min, 15 s and 15 s at 95° C., 60° C., 95° C. and 60° C.

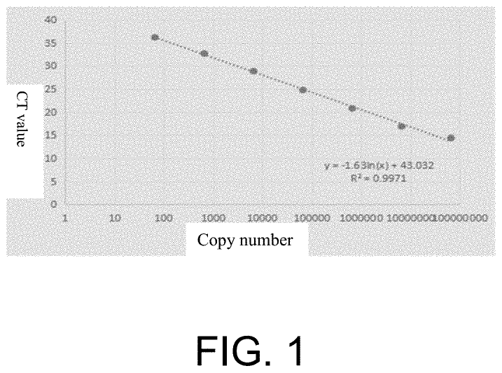

3. A standard curve was obtained according to CT results of different copy number of the plasmid, as shown in table 1 and FIG. 1, y=−1.63 ln(x)+43.032, R2=0.9971, as the CT value has reference significance within 15-35, the scope of DNA copy number capable of being detected by the present disclosure is within 1.2×102-3×107 copies.

TABLE 1
CT values of different copy number

| | 6.6 | 6.6 × 10 | 6.6 × 10² | 6.6 × 10³ | 6.6 × 10⁴ | 6.6 × 10⁵ | 6.6 × 10⁶ | 6.6 × 10⁷ | 6.6 × 10⁸ | 6.6 × 10⁹ | 6.6 × 10¹⁰ |
|---|---|---|---|---|---|---|---|---|---|---|---|
| Copy number | copies | copies | copies | copies | copies | copies | copies | copies | copies | copies | copies |
| CT value of | 38.8391 | 36.913 | 32.6028 | 29.3398 | 25.0715 | 21.0771 | 16.9501 | 14.4373 | 13.3028 | Undeter- | Undeter- |
| pore 1 | | | | | | | | | | mined | mined |
| CT value of | 29.029 | 36.423 | 32.9157 | 28.523 | 24.8861 | 20.7303 | 16.8347 | 14.4128 | 13.3069 | Undeter- | Undeter- |
| pore 2 | | | | | | | | | | mined | mined |
| CT value of | 37.5788 | 35.2617 | 32.566 | 28.7366 | 24.3641 | 20.5763 | 16.8018 | 14.3985 | 13.3466 | Undeter- | Undeter- |
| pore 3 | | | | | | | | | | mined | mined |
| Average CT | 35.1490 | 36.1990 | 32.6948 | 28.8664 | 24.7739 | 20.7945 | 16.8622 | 14.4162 | 13.3188 | | |
| value | | | | | | | | | | | |
| TM | 85.4 | 85.8 | 85.8 | 85.8 | 85.8 | 85.8 | 85.8 | 85.4 | 85.4 | 85.1 | 84.8 |

In the present disclosure, the plasmid pUC57 containing the sequence of 16S rDNA conserved region was taken to build a standard curve, and the scope of the DNA copy number capable of being detected by the present disclosure was within 1.2×10²-3×10⁷ copies. In the present disclosure, the amplification efficiency of the QPCR reaction was 86%, R2 was 0.9971.

III. Detection of Common Bacteria and Mixed Bacteria by Means of QPCR and Conventional PCR

1. Sample pretreatment: the sample to be detected was directly pretreated for 10 min at 100° C. and 12000 g samples were centrifuged for 5 min. The sample included Escherichia coli, Staphylococcus aureus, Bacillus subtilis, Pseudomonas aeruginosa, Salmonella, Streptococcus, degenerating Bacilus, Klebsiella pneumoniae and a mixture of the above 8 bacteria.

2. QPCR reaction system and conditions were the same as step 2 of II.

3. Preparation of conventional PCR system: total volume of the reaction system was 20 μl, the forward primer was 300 nM, reverse primer was 300 nM and the sample size was 1 μl. Reaction conditions: the first stage was a predegeneration stage for 30 s at 95° C., the second stage was a PCR reaction with 35 cycles for 20 s, 30 s and 30 s at 95° C., 58° C. and 72° C. respectively; the third stage was the extension of amplified products for 5 min at 72° C. 2% agarose gel was used for electrophoresis for 20 min at 120V. DL2000 was a Marker.

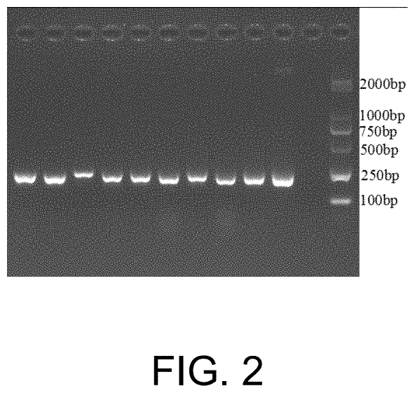

Detection results of the strains by conventional PCR are shown in FIG. 2, namely, Escherichia coli, Staphylococcus aureus, Bacillus subtilis, Pseudomonas aeruginosa, Salmonella, Streptococcus, degenerating Bacilus, Klebsiella pneumoniae, mixed bacteria, positive plasmid (2.17 ng/μl), negative control and Marker successively from left to right.

The present disclosure may detect all strains containing upstream and downstream primers by conventional PCR, including multiple common bacteria, e.g., Escherichia coli, Staphylococcus aureus, Bacillus subtilis, Pseudomonas aeruginosa, Salmonella, Streptococcus, degenerating Bacilus, Klebsiella pneumoniae and several mixed bacteria.

TABLE 2
Detection of different strains by QPCR

| Strains | CT value | TM |
|---|---|---|
| Escherichia coli | 15.4913 | 85.4 |
| Staphylococcus aureus | 14.018 | 84.5 |
| Bacillus subtilis | 20.2499 | 84.2 |
| Pseudomonas aeruginosa | 16.6841 | 85.8 |
| Salmonella | 16.6246 | 85.1 |
| Bacillus subtilis | 16.3097 | 84.8 |
| Degenerating bacilus | 14.5702 | 85.4 |
| Klebsiella pneumoniae | 16.2891 | 85.4 |
| Mixed bacteria | 15.1623 | 84.5 |
| Negative control | 37.6992 | 74.9 |

It can be seen from table 2 that the present disclosure may detect all strains containing upstream and downstream primers by fluorescent quantitative PCR, including multiple common bacteria, e.g., Escherichia coli, Staphylococcus aureus, Bacillus subtilis, Pseudomonas aeruginosa, Salmonella, Streptococcus, degenerating Bacilus, Klebsiella pneumoniae and several mixed bacteria.

IV. Detection of Human and Murine Cells by QPCR and Conventional PCR

Detection of Human and Murine Cells

Frozen human immune cells and rat stem cells were taken and washed by 3 times by PBS, then DNA was extracted by a DNA extraction kit and detected by the present disclosure, the detection method was the same as step 2 of II and step 3 of III.

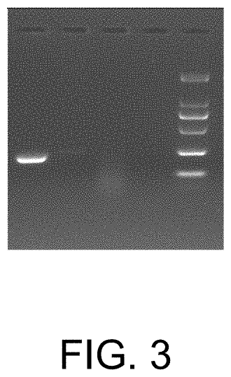

Detection results of human and murine cells by conventional PCR are shown in FIG. 3, namely, positive plasmid (2.17 ng/μl), human gene (96.6 ng/μl), murine gene (60.7 ng/μl), negative control and Marker successively from left to right.

In the method of the present disclosure, the sequence of the 16S rDNA conserved region had specificity, and it failed to detect the gene sequence of non-bacterial species, e.g., human immune cell, rat cell by conventional PCR.

TABLE 3
Detection of human and murine cells by QPCR

| Gene | CT value |
|---|---|
| Human | 37.0262 |
| Murine | Undetermined |
| Negative control | 38.2187 |
| Positive control | 16.9201 |

It can be seen from table 3 that the sequence of the 16S rDNA conserved region has specificity, and it fails to detect the gene sequence of non-bacterial species, e.g., human immune cell, rat cell by fluorescent quantitative PCR.

The foregoing descriptions are only preferred implementation manners of the present disclosure. It should be noted that for a person of ordinary skill in the art, several improvements and modifications may further be made without departing from the principle of the present disclosure. These improvements and modifications should also be deemed as falling within the protection scope of the present disclosure.

What is claimed is:

1. 1. A primer for rapid detection of bacteria, wherein a nucleotide sequence of a forward primer for the detection is shown in SEQ ID NO.1, and a nucleotide sequence of a reverse primer is shown in SEQ ID NO.2;
a degenerate base W=A/T in the forward primer, and a degenerate base R=A/G in the reverse primer.

2. A kit for rapid detection of bacteria, wherein the kit comprises the primer of claim 1 and a standard.

3. The kit according to claim 2, wherein the standard comprises a plasmid pUC57 containing a sequence of a 16S rDNA conserved region, and a nucleotide sequence of the sequence of the 16S rDNA conserved region is shown in SEQ ID NO.3.

4. The kit according to claim 2, wherein the service concentration of the primer is 100-400 nM.

5. The kit according to claim 2, Q-PCR reaction conditions of the kit being: 95° C., 10 min; 95° C., 20 s, 58° C., 30 s, 72° C., 30 s, 40 cycles; 95° C., 15 s, 60° C., 1 min, 95° C., 15 s, 60° C., 15 s.

6. The kit according to claim 2, conventional PCR reaction conditions of the kit being: 95° C., 10 min; 95° C., 20 s, 58° C., 30 s, 72° C., 30 s, 35 cycles; 72° C., 5 min.

7. An application of the kit according to claim 1 in the detection of bacteria.

8. An application of the kit according to claim 2 in the detection of bacteria.

9. An application of the kit according to claim 3 in the detection of bacteria.

10. An application of the kit according to claim 4 in the detection of bacteria.

11. An application of the kit according to claim 5 in the detection of bacteria.

12. An application of the kit according to claim 6 in the detection of bacteria.

13. An application of the kit according to claim 1 in the detection of bacteria of cells.

14. An application of the kit according to claim 2 in the detection of bacteria of cells.

15. An application of the kit according to claim 3 in the detection of bacteria of cells.

16. An application of the kit according to claim 4 in the detection of bacteria of cells.

17. An application of the kit according to claim 5 in the detection of bacteria of cells.

18. An application of the kit according to claim 6 in the detection of bacteria of cells.

19. An application of the kit according to claim 1 in the detection of bacteria of stem cells.

20. An application of the kit according to claim 2 in the detection of bacteria of stem cells.


SEQUENCE LISTING (3 sequences)

SEQ ID NO: 1 - DNA, 19 bp, artificial sequence
  a forward primer
cgawgcaacgcgaagaacc

SEQ ID NO: 2 - DNA, 18 bp, artificial sequence
  a reverse primer
caccttcctccrgtttrt

SEQ ID NO: 3 - DNA, 229 bp, Artificial sequence
  16S rDNA
attcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtgggg